SEQUENCE LISTING (4 sequences)

SEQ ID NO: 1 - DNA, 20 bp, Artificial Sequence
  Jagged-1 primer
gtgtctcaacgggggaactt

SEQ ID NO: 2 - DNA, 20 bp, Artificial Sequence
  Jagged-1 primer
acacaaggtttggcctcaca

SEQ ID NO: 3 - DNA, 20 bp, Artificial Sequence
  CXCL12 primer
tcagcctgagctacagatgc

SEQ ID NO: 4 - DNA, 20 bp, Artificial Sequence
  CXCL12 primer
tcagcctgagctacagatgc

(19) United States
(12) Patent Application Publication
(10) Pub. No.: US 2019/0201446 A1
(43) Pub. Date: Jul. 4, 2019

(54) HETEROGENEOUS NICHE ACTIVITY IN MESENCHYMAL STROMAL CELL-BASED CELL THERAPY

(71) Applicant: The Catholic University of Korea Industry-Academic Cooperation Foundation, Seoul (KR)

(72) Inventor: Il Hoan OH, Seoul (KR)

(21) Appl. No.: 16/091,709

(22) PCT Filed: Apr. 5, 2017
(86) PCT No.: PCT/KR2017/003737

§ 371 Date: Oct. 5, 2018

(30) Foreign Application Priority Data
Apr. 6, 2016 (KR) 10-2016-0042203

(51) Int. Cl. A61K 35/28; A61P 35/02; C12N 5/0775

(52) U.S. Cl. CPC A61K 35/28; A61P 35/02; C12N 5/0668; C12N 5/0663; C12N 5/0667

(57) ABSTRACT

The present invention relates to heterogeneous niche activity in mesenchymal stromal cell-based cell therapy. It was discovered in the present invention that a difference in the niche activities of MSCs can be created during ex-vivo expansion of MSCs to cause a variation in the outcomes of hematopoietic recoveries. Particularly, the difference in caused by the functional state of MSCs derived by distinct upstream signaling pathways, rather than by clonal heterogeneity, and the functional state can be inferred through the CFU-F of MSC. Therefore, the present invention is expected to contribute to solving a variation in therapeutic effects which is pointed out as a problem of conventional mesenchymal stromal cell-based cell therapy.

### CROSS REFERENCE TO RELATED APPLICATION

This application is a Section 371 of International Application No. PCT/KR2017/003737, filed Apr. 5, 2017, which was published in the Korean language on Oct. 12, 2017 under International Publication No. WO 2017/176048 Al, which claims priority under 35 U.S.C. § 119(b) to Korean Application No. 10-2016-0042203, filed Apr. 6, 2016, the disclosures of which are incorporated herein by reference in their entirety.

### REFERENCE TO SEQUENCE LISTING SUBMITTED ELECTRONICALLY

This application contains a sequence listing, which is submitted electronically via EFS-Web as an ASCII formatted sequence listing with a file name “688588.25 Sequence Listing” and a creation date of Mar. 21, 2019, and having a size of 1.1 KB. The sequence listing submitted via EFS-Web is part of the specification and is herein incorporated by reference in its entirety.

### BACKGROUND

### 1. Field of the Invention

The present invention relates to heterogeneous niche activity in mesenchymal stromal cell-based cell therapy.

### 2. Discussion of Related Art

Mesenchymal stem cells (MSCs) are non-hematopoietic adherent cell populations derived from bone marrow (BM), adipose tissue, or placental tissue, and exhibit multi-lineage differentiation potency. Recent studies have shown that the major action of MSCs is the paracrine function of helping tissue regeneration by inhibiting apoptosis and fibrosis and stimulating the regeneration of endogenous stem cells such as hematopoietic stem cells (HSCs), neuronal stem cells, and other tissue-specific stem cells.

MSCs present in bone marrow constitute perivascular and endosteal niches. A part of MSCs that retain colony-forming potential (CFU-F) and self-renewing capacity can reconstitute both types of niches in a heterologous bone marrow model. The niche cells express various types of growth factors or ligands such as Jagged-1 or CXCL-12 to regulate self-renewal or quiescence of HSCs. Recently, it was shown that physiological stimuli can stimulate niche activities of MSC subpopulations, and thereby induce HSCs to reversibly switch between dormant and activated states (Korean Unexamined Patent Application No. 2009-0008155). Similarly, the inventors showed that regulation of the niche activity of MSCs may be a very critical factor for regulating the regenerative activity of HSCs, and that functional changes in MSCs are regulated to heterogenous clinical prognosis in hematological malignant tumors. In other words, the niche activity of MSCs can exert a significant impact on the regeneration of HSCs.

However, MSCs are generally prepared by ex-vivo culture with a fetal bovine serum (FBS) supplement, and the culture-expanded MSCs undergo functional and phenotypic changes exhibiting different characteristics from in vivo-isolated MSCs. Moreover, various aspects of clonal heterogeneity have been observed among ex-vivo expanded MSC populations with respect to their morphology, proliferation, multi-lineage differentiation and self-renewing potential. Therefore, ex-vivo expanded MSCs are prone to heterogeneity by selective expansion of clones or functional changes during culture. In animal model experiments using in vitro co-culture of murine or human HSCs, despite the complex heterogeneity in MSC subpopulations, ex-vivo expanded MSCs showed supportive activities for HSCs.

While successive results from such clinical experiments did not show evidence of toxicity, clinical results are highly variable regardless of the source for HSCs used in transplantation. For example, a variety of studies reported that a reduced graft failure rate is caused by acceleration of leukocyte recovery after co-transplantation with MSCs, whereas other studies reported no beneficial effect on engraftment and hematopoietic recovery. Therefore, identification of the fundamental causes of various therapeutic effects in MSC-based cell therapy is of major interest.

### SUMMARY OF THE INVENTION

The present invention is provided to solve the above-mentioned problems, and the inventors have attempted to improve the effectiveness of mesenchymal stromal cell-based cell therapy, and thus confirmed that the niche activity of MSCs or the supporting activity with respect to HSCs is reversibly changed according to ex-vivo culture conditions, not individual differences, and the niche activity can be expected through colony-forming unit fibroblasts (CFU-Fs) of cultured MSCs, and based on this, the present invention was accomplished.

The present invention is directed to providing a method of screening MSCs with improved niche activity.

The present invention is also directed to providing a composition for promoting self-renewal of HSCs, which contains the selected MSCs.

However, technical problems to be solved in the present invention are not limited to the above-described problems, and other problems which are not described herein will be fully understood by those of ordinary skill in the art from the following descriptions.

To attain the object of the present invention, the present invention provides a method of screening MSCs with improved niche activity, which includes: ex-vivo culturing isolated MSCs; and selecting MSCs in which 10% or more of a total of the cultured MSCs form CFU-Fs.

In addition, the present invention provides a method of screening culture conditions to improve the niche activity of MSCs, which includes: assessing the CFU-F number of ex-vivo cultured MSCs; and selecting culture conditions under which 10% or more of a total of the cultured MSCs form CFU-Fs.

In one exemplary embodiment of the present invention, the MSCs may be passaged two to five times.

In another exemplary embodiment of the present invention, the CFU-F number may be assessed 10 to 17 days after cultured stem cells are plated.

In still another exemplary embodiment of the present invention, the MSCs may be derived from human adipose tissue, bone marrow, peripheral blood or umbilical cord blood.

In yet another exemplary embodiment of the present invention, the niche activity may support the undifferentiating capacity of HSCs, and stimulate self-renewing capacity.

In addition, the present invention provides a composition for stimulating self-renewal of HSCs, which includes the selected MSCs.

In one exemplary embodiment of the present invention, the composition may be used for transplantation into a patient with acute leukemia, chronic myelogenous leukemia, myelodysplastic syndrome, lymphoma, multiple myeloma, a germ cell tumor, breast cancer, ovarian cancer, small cell lung cancer, neuroblastoma, aplastic anemia, erythropathy, Gaucher's disease, Hunter syndrome, adenosine deaminase (ADA) deficiency, Wiskott-Aldrich syndrome, rheumatoid arthritis, systemic lupus erythematosus, or multiple sclerosis, or a patient with damaged hematopoietic cells due to chemotherapy or radiation therapy.

In another exemplary embodiment of the present invention, the composition may be transplanted together with HSCs, and the HSCs may have Lin⁻Sca-1⁺c-kit⁺ (LSK) as a marker for a primitive undifferentiated state.

In addition, the present invention provides a method of stimulating self-renewal of HSCs, which includes co-culturing the selected MSCs and HSCs.

The present invention showed that a difference in the niche activity of MSCs is made in an ex-vivo expansion step, resulting in various results in hematopoietic recovery. Particularly, such a difference was caused by the functional state of MSCs induced by inherent upstream signaling pathways, rather than clonal heterogeneity, and such a functional state was able to be inferred by CFU-F contents in MSCs. Therefore, it is expected that the present invention contributes to resolution of variability of therapeutic effects which had been indicated as a problem of mesenchymal stromal cell-based cell therapy.

### BRIEF DESCRIPTION OF THE DRAWINGS

FIG. 1a is a schematic diagram illustrating an experimental process for screening a stimulatory (SS-1, SS-2) or non-stimulatory (NSS-1, NSS-2) medium using a medium supplemented with fetal bovine serum (FBS).

FIG. 1b shows the result of comparing CFU-F numbers of MSCs derived from various donors, which are cultured in a stimulatory (SS-1, SS-2) or non-stimulatory (NSS-1, NSS-2) medium.

FIG. 1c shows the result of comparing CFU-F numbers in high-proliferating colonies (large colony; >4 mm) or low-proliferating colonies (small colony; <4 mm), which are cultured in a stimulatory (SS-1, SS-2) or non-stimulatory (NSS-1, NSS-2) medium.

FIG. 1d shows the result of comparing doubling times of MSCs cultured in a stimulatory (SS) or non-stimulatory (NSS) medium.

FIG. 1e shows the result of comparing changes in surface phenotypes (CD34, CD271, CD166, CD146, CD140a, SSEA4, CD73) of MSCs cultured in a medium with stimulatory (SS) and non-stimulatory (NSS) conditions.

FIG. 1f shows the morphological characteristic of MSCs cultured in a stimulatory (SS) or non-stimulatory (NSS) medium, observed using an optical microscope.

FIG. 1g shows the result of comparing the physical properties of MSCs cultured in a medium with stimulatory (SS) and non-stimulatory (NSS) conditions, obtained by flow cytometry analysis.

FIG. 1h shows the result of comparing osteogenic (left) and adipogenic (right) differentiation of MSCs cultured in a medium with stimulatory (SS) and non-stimulatory (NSS) conditions.

FIG. 2a is a RT-PCR result of comparing Jagged-1 and CXCL-12 gene expression levels of MSCs cultured in a medium with stimulatory (SS) and non-stimulatory (NSS) conditions.

FIG. 2b is a flow cytometric result of comparing Jagged-1 and CXCL-12 positive cells (%) of MSCs cultured in a medium with stimulatory (SS) and non-stimulatory (NSS) conditions.

FIG. 3a is a schematic diagram illustrating an experimental process for confirming changes in the supporting activity of HSCs after MSCs cultured in a medium with stimulatory (SS) and non-stimulatory (NSS) conditions are co-cultured with UCB-derived CD34⁺ cells.

FIG. 3b shows the result of comparing changes in the number of colony-forming cells (CFCs) after MSCs cultured in a medium with stimulatory (SS) and non-stimulatory (NSS) conditions are co-cultured with UCB-derived CD34⁺ cells for 5 days.

FIG. 3c shows the result of comparing the total number of CD34⁺/CD90+ cells after MSCs cultured in a medium with stimulatory (SS) and non-stimulatory (NSS) conditions are co-cultured with UCB-derived CD34⁺ cells for 5 days.

FIG. 3d shows the result of comparing changes in the number of CFCs after MSCs cultured in a medium with stimulatory (SS) and non-stimulatory (NSS) conditions are co-cultured with UCB-derived CD34⁺ cells for 6 weeks.

FIG. 4a shows the result of comparing the CFU-F number of murine MSCs cultured in a medium with stimulatory (SS) and non-stimulatory (NSS) conditions.

FIG. 4b shows the RQ-PCR result of comparing Jagged-1 and SDF-1 gene expression levels of murine MSCs cultured in a medium with stimulatory (SS) and non-stimulatory (NSS) conditions.

FIG. 4c shows the flow cytometric result of comparing Jagged-1 and SDF-1 positive cells (%) of murine MSCs cultured in a medium with stimulatory (SS) and non-stimulatory (NSS) conditions.

FIG. 4d shows the result of quantitatively comparing Jagged-1 and SDF-1 positive cells (%) of murine MSCs cultured in a medium with stimulatory (SS) and non-stimulatory (NSS) conditions.

FIG. 5a is a schematic diagram illustrating an experimental process for confirming changes in the supporting activity of HSCs after murine MSCs cultured in a medium with stimulatory (SS) and non-stimulatory (NSS) conditions are co-cultured with HSCs.

FIG. 5b shows the result of comparing donor-derived cells (45.1+ cell %) at 9 or 12 weeks after murine MSCs cultured in a medium with stimulatory (SS) and non-stimulatory (NSS) conditions are co-transplanted with HSCs (45.1+) (priming: transplantation after 2-hour mixing, direct: direct transplantation into a recipient without pretreatment).

FIG. 5c shows the result of comparing lineage distribution of donor-derived leukocytes present in peripheral blood of a recipient at 12 weeks after murine MSCs cultured in a medium with stimulatory (SS) and non-stimulatory (NSS) conditions are co-transplanted along with HSCs (45.1+).

FIG. 5d shows the result of comparing the number of donor-derived Lin⁻Sca-1⁺c-kit⁺ (LSK) cells at 12 weeks after murine MSCs cultured in a medium with stimulatory (SS) and non-stimulatory (NSS) conditions are co-transplanted along with HSCs (45.1+).

FIG. 6a is a schematic diagram illustrating an experimental process for confirming changes in the reversible niche activity of MSCs cultured in a stimulatory (SS) or non-stimulatory (NSS) medium.

FIG. 6b shows the result of confirming the CFU-F number of MSCs cultured by switching between stimulatory (SS) and non-stimulatory (NSS) conditions.

FIG. 6c shows the result of confirming the number of primitive hematopoietic cell populations (CD34⁺/CD90+ cells) after MSCs cultured in a third medium with stimulatory (SS) and non-stimulatory (NSS) conditions are co-cultured along with CD34⁺ cells.

FIG. 6d is the result of confirming the number of primitive hematopoietic cell populations (CD34⁺/CD90⁺ cells) after MSCs cultured by switching between stimulatory (SS) and non-stimulatory (NSS) conditions are co-cultured with CD34⁺ cells.

FIG. 7a is a microarray plot for the signal pathways of MSCs cultured in a stimulatory (SS) or non-stimulatory (NSS) medium.

FIGS. 7b to 7g are gene set enrichment analysis (GSEA) results for the signaling pathways of MSCs cultured in a stimulatory (SS) or non-stimulatory (NSS) medium.

### DETAILED DESCRIPTION OF EXEMPLARY EMBODIMENTS

Hereinafter, the present invention will be described in detail.

The present invention provides a method of screening MSCs with improved niche activity, which includes: ex-vivo culturing isolated MSCs; and selecting MSCs in which 10% or more of a total of the cultured MSCs form CFU-Fs.

In addition, the present invention provides a method of screening a culture condition to improve the niche activity of MSCs, which includes: assessing the CFU-F number of ex-vivo cultured MSCs; and selecting culture conditions under which 10% or more of a total of the cultured MSCs form CFU-Fs.

The term “mesenchymal stem cells (MSCs)” used herein are cells which serve as the origin for creating cartilage, bone, fat, bone marrow stroma, muscle, nerve, etc., and in adults, are generally present in the bone marrow, but also present in umbilical cord blood, peripheral blood, and other tissues, and thus are obtained therefrom. In the specification, MSCs are used in the same sense as mesenchymal stromal cells or stromal cells. The MSCs include cells derived from all animals such as humans, monkeys, pigs, horses, cows, sheep, dogs, cats, mice, and rats, and preferably human-derived cells.

The term “niche” used herein refers to a component (cells and/or a material) consisting of tissues or organs supporting development and proliferation of tissue cells such as stem cells and somatic cells, other than the stem cells, and the niche has been known to secrete factors required for inducing cellular interactions and having totipotency. The niche is also called a microenvironment, and plays a critical role in retaining stemness expressing all characteristics of stem cells. Stem cells are anchored in a type of microenvironment consisting of adhesion molecule growth factors, which is called niche in the academic circles. Such a region of a stem cell serves to support and regulate a location, adhesiveness, homing, quiescence, and activation. In other words, the niche is considered as a major microenvironment that surrounds stem cells serving to regulate differentiation of stem cells, and prevent and protect migration to another site or apoptosis. Particularly, the actively-studied field of stem cell niches is an HSC niche, and the HSC niche of the present invention is the place where HSCs reside. The HSCs, which are a type of stem cell, can differentiate into all types of blood cells, but if away from their own niche, that is, the HSC niche, cannot properly exhibit the above-mentioned ability. Like other niches, it has been known that the HSC niche is not only a simple shelter for HSCs, but also regulates the number of corresponding stem cells, that is, HSCs. For the purpose of the present invention, the niche activity may mean an ability of supporting the undifferentiating capacity of HSCs and stimulating self-renewing capacity.

The term “self-renewal” used herein refers to an ability of producing cells having the same properties and characteristics, is also called self-replication or self-reproduction, and one of the critical characteristics of stem cells. Particularly, in the present invention, the self-renewal means an ability of continuing proliferation while an undifferentiated state is maintained.

Meanwhile, ex-vivo expanded MSCs have been widely used as a paracrine support material for restoration of hematopoietic function, and have difficulty in clinical application due to inconsistent efficacy. Therefore, to improve the effectiveness of MSCs-based cell therapy, screening of MSCs with improved niche activity is a very important technical task.

The inventors selected CFU-Fs of ex-vivo expanded MSCs as a parameter for niche activity (10% or more CFU-Fs) to classify stimulatory or non-stimulatory medium conditions, and thus the difference in effects was observed. As a result, in MSCs sub-cultured under stimulatory conditions, expression of cross-talk molecules (Jagged-1 and CXCL-12) was improved, and the enhancing effect of MSCs on hematopoietic engraftment or recovery was observed only when MSCs cultured under stimulatory conditions are co-cultured along with HSCs. Particularly, such an effect reversibly switches by reversing medium conditions, first indicating that the difference in niche activity can be caused by a functional state of MSCs, that is, a change in culture conditions, rather than by clonal heterogeneity. Actually, it was confirmed that the MSCs cultured under stimulatory conditions, unlike those cultured under non-stimulatory conditions, have intrinsic signaling pathways such as inhibition of p53 and activation of ATF, and therefore it was reconfirmed that the niche activity of MSCs is determined by extrinsic factors during in-vitro culture. Based on such experimental results, the present invention has technical characteristics in that the quality of a cell therapeutic agent can be standardized and improved by selecting MSCs with improved niche activity, and new culture conditions for improving the niche activity of MSCs can be screened.

In the present invention, for quantitative evaluation of niche activity, the MSCs may be passaged 2 to 5 times, and most preferably 2 times, and the calculation or evaluation of the CFU-F number may be performed 10 to 17 days, and most preferably 14 days after the cultured MSCs are plated in a medium.

In the present invention, culture conditions are preferably a supportive material added to a medium, but may also include physical stimulation, physiological changes (hypoxic state, expression of a specific factor, etc.), the change of a culture method (three-dimensional culture), etc.

In another aspect of the present invention, a composition for stimulating self-renewal of HSCs, which includes the selected MSCs; a method of stimulating self-renewal of HSCs, which includes co-culturing the selected MSCs and HSCs; and a method of stimulating self-renewal of HSCs, which includes administering the selected MSCs and HSCs to a subject, are provided.

Since the composition for stimulating self-renewal of HSCs of the present invention uses MSCs which have been described in the above-described method of screening MSCs with improved niche activity, common descriptions will be omitted to avoid excessive complexity of the specification.

The term “hematopoietic stem cells (HSCs)” used herein refers to the ancestor cells of undifferentiated bone marrow hematopoietic cells which produce blood cells such as erythrocytes, leukocytes, platelets, etc., and exhibits an ability of long-term repopulation with self-renewing capacity when being transplanted into a bone marrow-destroyed host. The HSCs include cells derived from all animals such as humans, monkeys, pigs, horses, cows, sheep, dogs, cats, mice, rats, etc., and preferably, human-derived cells. As an example, the HSCs may have LSK as an indicator for a primitive undifferentiated state.

The composition of the present invention is transplanted along with a therapeutically effective amount of hematopoietic stem cells in patients in a physiological state in which HPCs are damaged. The physiological state in which hematopoietic cells are damaged may be caused by acute leukemia, chronic myelogenous leukemia, myelodysplastic syndrome, lymphoma, multiple myeloma, germ cell tumors, breast cancer, ovarian cancer, small cell lung cancer, neuroblastoma, aplastic anemia, erythropathy, Gaucher's disease, Hunter syndrome, ADA deficiency, Wiskott-Aldrich syndrome, rheumatoid arthritis, systemic lupus erythematosus or multiple sclerosis, or chemotherapy or radiation therapy.

Hereinafter, exemplary examples will be presented to help in understanding of the present invention. However, the following examples are merely provided to more easily understand the present invention, and the scope of the present invention is not limited by the following examples.

### Example 1. Experimental Materials and Preparation

### 1-1. Umbilical Cord Blood, MSCs, and Ex-Vivo Culture

Umbilical cord blood (CB) was obtained from healthy pregnant woman donors under written consent. In this study, the written consent and all experiments were approved by the Institutional Review Board of the Catholic University of Korea (CUMC11U077). In addition, MSCs were also obtained under written consent from healthy donors under approval from the Institutional Review Board of the Catholic University of Korea (KC13MDMS0839). For donors under the age of 18, the written consent was obtained from their parents, instead of the donors (MC12TNSI0120).

MSC cultures were established from BM mononuclear cells, and passaged in the Dulbecco's modified Eagle's medium (DMEM) containing 10% fetal bovine serum (FBS). During culture, different FBS batches were purchased and tested for their effects on MSCs. Culture of MSCs under hypoxic conditions was performed in a CO₂ water-jacketed hypoxic incubator (Thermo Fisher, Heracell 150i, Waltham, Mass.) adjusted to 1% O2.

To screen FBS batches, 7 and 5 randomly selected FBS batches were collected from different vendors (Gibco or Hyclone), and tested for effects on MSCs in two sets of independent screening. All FBS batches were cell culture grade, filtered (3×, 100 nm filter) and free of endotoxins, viruses or mycoplasma. To compare expansion in each different culture condition, MSCs were sub-cultured at least two passages before analysis. Doubling times were calculated at t/n, wherein t is the duration of culture, and n is the number of doubling individuals calculated by the equation n=log (NH−NI)/log2 (where NI is the number of cells originally plated; NH is the number of cells harvested at the time of counting).

### 1-2. Experimental Animals and In-Vivo Repopulation

Animal experiments were undertaken with approval from the Animal Experiment Board and the Institutional Review Board of the Catholic University of Korea. In congenic transplantation, C57BL/6J-Ly 5.2 (BL6) or C57BL/6J-Pep3b-Ly5.1 (Pep3b) mice were used as recipients or donors. Enrichment of murine bone marrow cells by 5-fluorouracil treatment (5-FU BMC) was performed by a conventional method. Murine MSCs were obtained from murine bone marrow by serial passage of adherent cells in a medium containing 10% FBS until the cells reached CD45 negative. BMC transplantation into lethally irradiated (900 rad) congenic recipient mice was performed by a conventional method. Co-transplantation studies were performed by either simultaneous co-injection of HSCs and MSCs into mice (direct) or injection of a mixture thereof which had been mixed in the same test tube 2 hours before injection (priming). The repopulation of the bone marrow was assessed by measuring the proportion of donor-derived CD45.1⁺ leukocytes (WBC) in peripheral blood samples. The lineage of the repopulated hematopoietic cells was analyzed by immunostaining; and anti-Mac-1/Gr-1 antibodies (BD Pharmingen, San Diego, Calif.) and anti-B220 antibodies (BD Pharmingen) were used to identify myeloid or B-lymphoid cells, respectively.

Mice were sacrificed 9 to 12 weeks after transplantation, and donor-derived cells were used to assess repopulation levels, and hierarchy analysis was performed by flow cytometry using antibodies against CD45 (BD Pharmingen), lineage markers (Stem Cell Technologies, Vancouver, BC, Canada), Sca-1-PEcy7 (BD Pharmingen), and c-kit-APC (eBioscience, San Diego, Calif., USA).

### 1-3. Flow Cytometry of MSCs

Flow cytometry for MSC surface markers was performed. Cells were stained with monoclonal antibodies, anti-human CD73-PE, CD-34-APC, CD146-PEcy7, CD271-APC, Streptavidin-PEcy7, CD140a-PE (BD Pharmingen), SSEA4-Biotin (R&D Systems, Minneapolis, Minn.), and CD166-FITC (Serotec, Oxford, UK), and analyzed using FACSCalibur (Becton Dickinson) and CellQuest software. To examine the expression of cross-talk molecules, MSCs were permeabilized, and intracellularly-stained with Jagged-1-specific antibodies (28H8, Cell Signaling, Danvers, Mass.) or CXCL-12-specific antibodies (79018, R&D Systems). Relative expression levels were determined by ΔMFI, which is a difference in mean fluorescence intensity. Osteogenic and adipogenic differentiation of MSCs were induced in specific differentiation media, respectively, and quantified by Alizarin Red staining or lipid droplets. For colony formation (CFU-F), MSCs were plated at a density of 500 cells per 100 mm dish and incubated for 14 days, and the number of colonies was counted after staining with crystal violet in a methanol solution.

### 1-4. RT-PCR and RQ-PCR

For RT-PCR analysis, RNA was purified from MSCs, and cDNA was prepared from the RNA using random hexamers and SuperScript II (Invitrogen, Carlsbad, Calif., USA) and amplified using specific primers for Jagged-1 (5′-GTG TCT CAA CGG GGG AAC TT-3′ (SEQ ID NO: 1) and 5′-ACA CAA GGT TTG GCC TCA CA-3′ (SEQ ID NO: 2)) or CXCL12 (5′-TCA GCC TGA GCT ACA GAT GC-3′ (SEQ ID NO: 3) and 5′-TCA GCC TGA GCT ACA GAT GC-3′ (SEQ ID NO: 4)). Real-time quantitative PCR (RQ-PCR) was performed with the Rotor-Gene 6000 system (Corbett Life Science, Australia) and SYBR premix Ex taq (Takara, Japan). After normalization to an endogenous GAPDH control group, relative expression levels of PCR products were determined. The threshold cycle (Ct) value for each gene was normalized to the Ct value of glyceraldehyde-3-phosphate dehydrogenase (GAPDH). Relative mRNA expression was calculated using the formula 2^{-ΔΔ}^{^{Ct}}, wherein ΔCt=Ctₛₐₘₚₗₑ−Ct_{ΔGAPDH} and ΔΔCt=ΔCtₛₐₘₚₗₑ−ΔCt_{reference group.}

### 1-5. Purification, Ex-Vivo Culture and Long-Term Culture of Hematopoietic Cells

CD34⁺ cells were purified from mononuclear cells of UCB by immunomagnetic cell separation (Dynabeads; Invitrogen, https://www.thermofisher.com). The cells were cultured in DMEM containing 100 ng/ml human Flt-3 ligand (Prospec Tany, Rehovot, Israel), 100 ng/ml human SCF (Prospec Tany), 40 ng/ml human IL-6 (R&D Systems), 40 ng/ml human IL-3 (R&D Systems), and 40 ng/ml human G-CSF (Prospec Tany) supplemented with 10⁻⁶M hydrocortisone sodium hemisuccinate (Sigma) to which each batch of fetal bovine serum (FBS) was added. Before co-culture, MSCs were irradiated (1500 cGy), and co-cultured along with the CD34⁺ cells for 5 days under the similar medium conditions. For colony forming assay of hematopoietic progenitor cells, hematopoietic cells were cultured in a cytokine-containing semi-solid methylcellulose medium (MethoCult; Stem Cell Technologies) for 14 days, and analyzed for colony numbers and lineages as described above. For long-term culture-initiating cell (LTC-IC) analysis, CD34⁺ cells were co-cultured with normal MSCs for 5 days, transferred to a medium for 6-week long-term culture, and subjected to a colony-forming assay in a semi-solid medium.

### 1-6. Microarray Analysis

An RNA extract was continuously amplified and hybridized to an oligonucleotide DNA microarray. A double-stranded DNA template was amplified by an Eberwine method which is a modified form of the T7 RNA polymerase-based linear amplification protocol using the T7 MEGAscript kit (Ambion, Austin, Tex.). A biotin-labeled cRNA sample was hybridized to Illumina Human HT-12 V4-BeadChip (48 K) (Illumina, Inc., San Diego, Calif.). Arrays were scanned, and the processing and analysis for the array results were performed using Illumina BeadStudio software. The microarray studies were performed by the Shared Research Equipment Assistance Program of the Korea Basic Science Institute (MEST).

Hierarchical clustering was performed using the Pearson correlation coefficient indicating the average relationship as a distance measure. The gene ontology (GO) program (http://david.abcc.ncifcrfgov/) was used to classify genes in functional subgroups, and gene set enrichment analysis (GSEA) was performed. In the GSEA, Kolmogorov-Smirnov statistics were used to calculate the significance level of enrichment for an up-regulating gene in MSCs under stimulatory conditions versus MSCs under non-stimulatory conditions. To identify up-regulating gene candidates which can cause gene transcriptomic changes in MSCs, ingenuity pathway analysis (IPA, Ingenuity Systems, www.ingenuity.com) was performed.

### 1-7. Statistical Analysis

To determine significance at a transcriptome level, a t-test was used to identify the difference in significance between MSCs (p<0.01). The overlapping P-value in the up-regulating genes was estimated by a Fish's exact test, and used to statistically measure the overlap significance between genes in the test result and genes regulated by a regulatory gene. An activation Z-score was estimated using target signal intensities of each regulatory gene to examine whether the up-regulating genes can be activated (positive score) or inhibited (negative score) during the pathway analysis.

### Example 2. Screening of MSCs Cultured Under Different Serum Conditions According to Heterogeneous CFU-F Content

The inventors hypothesized that heterogeneity in the stem cell-supporting activity of MSCs is created according to differences in culture conditions during the ex-vivo expansion of MSCs. Particularly, by using FBS which had been widely used as a supplement for a culture medium, the effect on the functional heterogeneity of MSCs was examined.

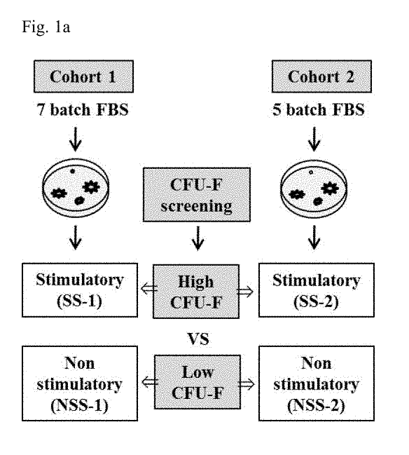

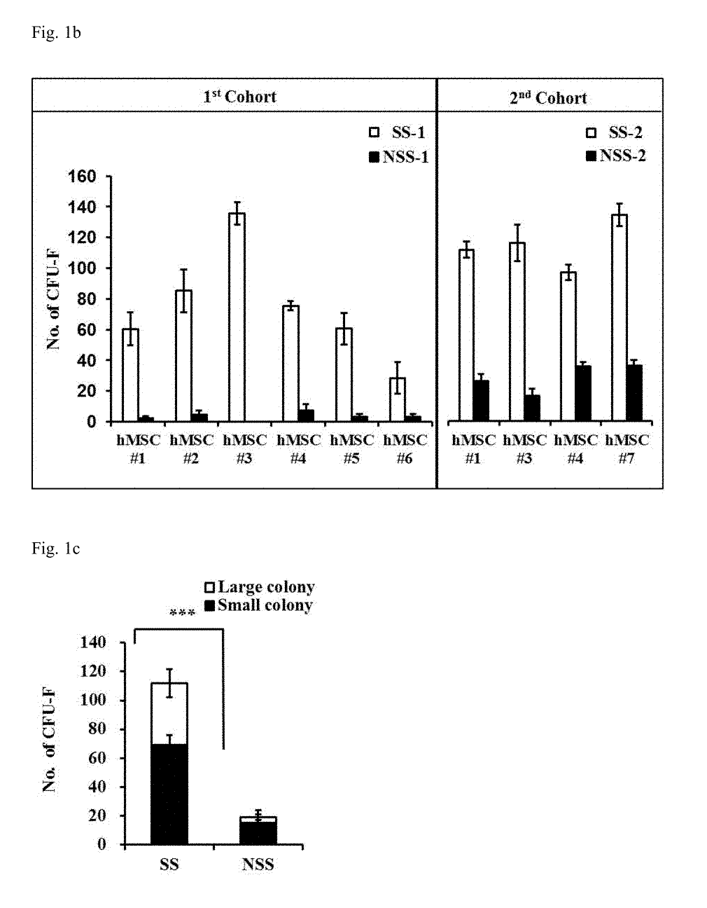

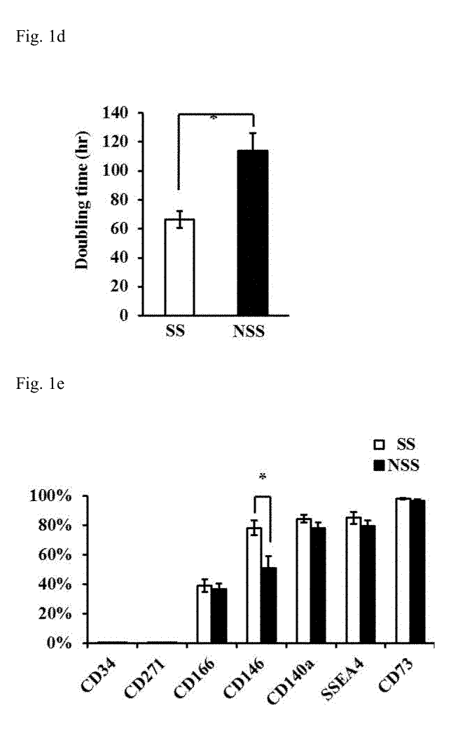

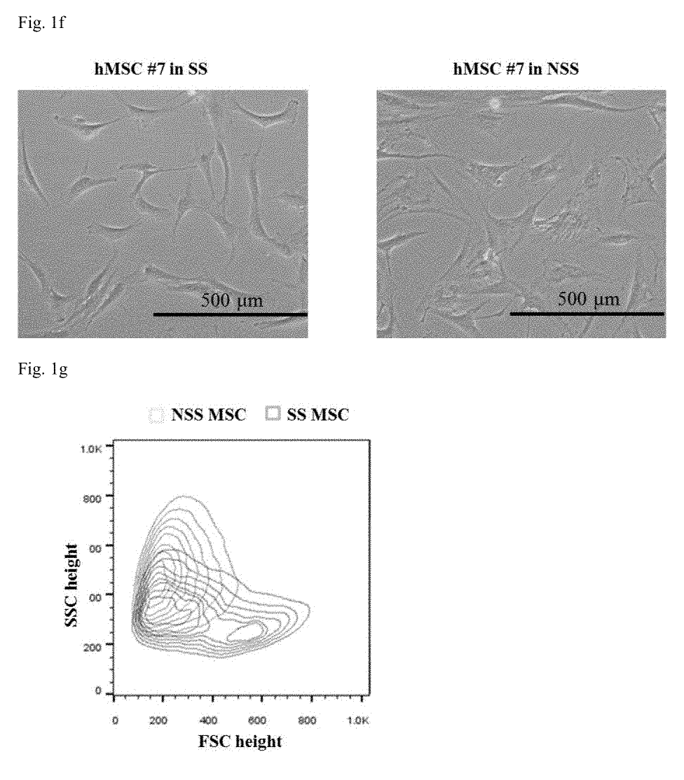

First, the inventors chose the frequency of CFU-Fs as a parameter for the heterogeneity of cultured MSCs, based on the fact that MSC subpopulations with enriched CFU-Fs serve as niche cells in bone marrow. From a test for two cohorts with respect to multiple FBS batches, two pairs of serum batches (stimulatory: SS-1 and SS-2 [when CFU-Fs>50 at 14 days after a total of 500 MSCs were plated] that can produce many (stimulatory) or less (non-stimulatory) CFU-Fs, non-stimulatory: NSS-1 and NSS-2 [when CFU-Fs<50 at 14 days after a total of 500 MSCs were plated]) were classified (FIG. 1a).

Such stimulatory (SS) or non-stimulatory (NSS) serum batches caused significant differences in CFU-Fs in a test for MSCs independently derived from 7 normal donors (FIG. 1b). These results indicate that the difference in serum batches, rather than variations in donor individuals, has a higher influence on CFU-Fs. In addition, as a result of analyzing respective effects on high (large colony)- or low (small colony)-proliferating colonies, the difference in serum batches had a similar influence on both types of colonies (FIG. 1c), and the doubling time of MSCs under stimulatory serum conditions was shorter than that under non-stimulatory conditions, and MSCs under the stimulatory serum conditions showed high proliferating activity (FIG. 1d). Meanwhile, when surface phenotypes were compared, the MSCs under the stimulatory conditions exhibited a higher proportion of CD146⁺ cells than those under the non-stimulatory conditions, and in the others, the proportion of CD271, CD140a, SSEA4, or CD73 were similarly observed (FIG. 1e). In addition, under an optical microscope, the MSCs under the stimulatory conditions were observed in a spindle shape, whereas the MSCs under the non-stimulatory conditions were observed in a more flattened shape (FIG. 1f), and as assessed with forward/side scatter in flow cytometry, unique profiles for the physical properties were shown (FIG. 1g). However, there was no significant difference in the osteogenic and adipogenic differentiations of MSCs depending on the stimulatory or non-stimulatory conditions (FIG. 1h).

### Example 3. Confirmation of Changes in Hematological Recovery Caused by Niche Activity of MSCs

In this example, the niche activity of MSCs cultured under stimulatory and non-stimulatory conditions in Example 2, which is associated with the support of HSCs, and the hematopoietic recovery effect caused thereby were compared.

3-1. Confirmation of Activity of Supporting HSCs Through Co-Culture with CD34⁺ Cells

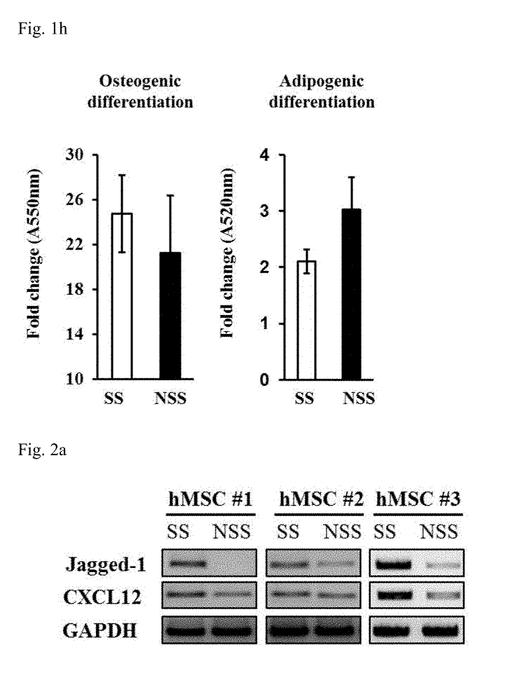

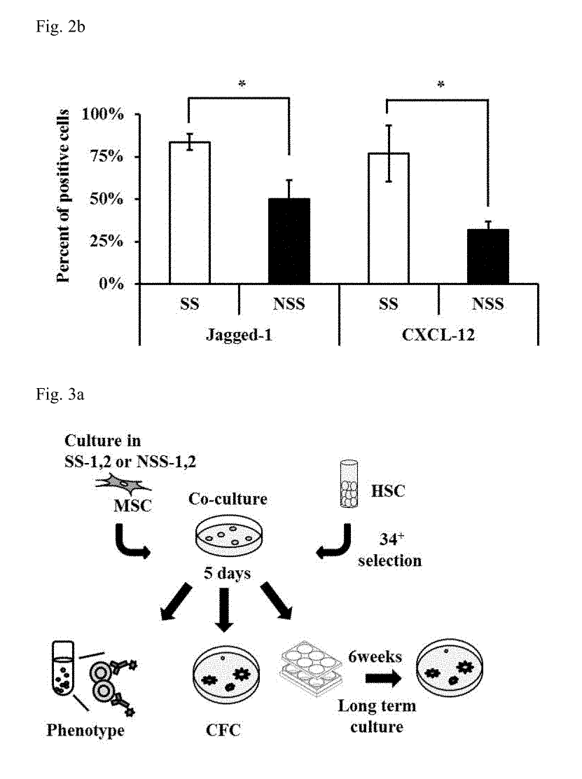

First, the inventors compared the expression levels of two cross-talk molecules known to play major roles in supporting HSCs under in-vivo and in-vitro conditions, Jagged-1 and CXCL-12. MSCs cultured under stimulatory conditions exhibited higher Jagged-1 and CXCL-12 expression levels than those cultured under non-stimulatory conditions (FIGS. 2a and 2b). This MSC-related result suggests that there is a possibility of niche cross-talk difference for HSCs.

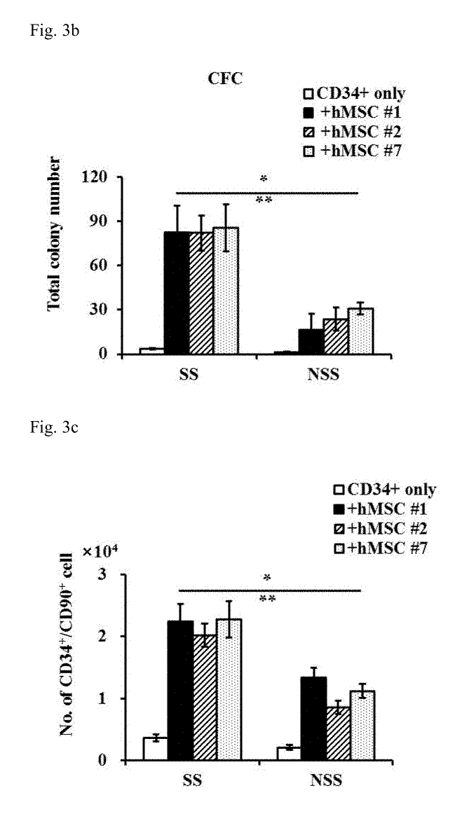

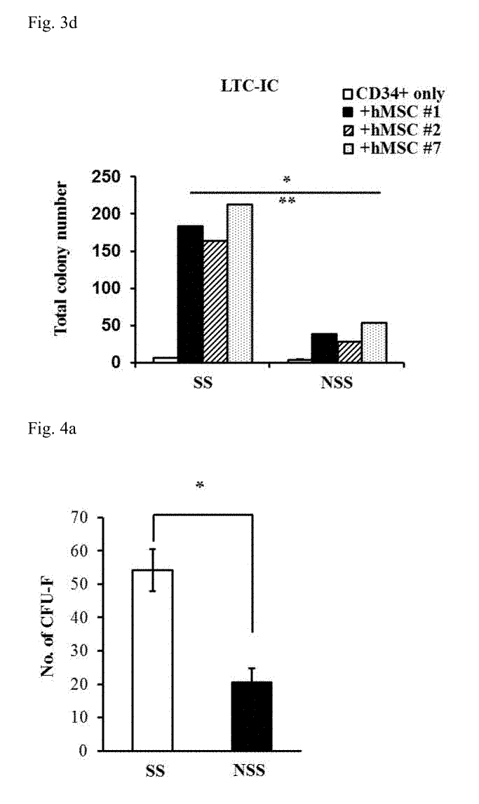

To examine such possibility, the inventors compared the HSC supporting activity of each group of MSCs (SS-1,2 or NSS-1,2) after being co-cultured with UCB-derived human CD34+ cells for 5 days (FIG. 3a). Each group of individual donor-derived MSCs expanded under stimulatory conditions (hMSC #1, #2 and #7) exhibited high supporting activities with respect to HSCs, confirmed by high CFU numbers (FIG. 3b). Further, MSCs cultured under stimulatory conditions showed high supporting activity of a primitive compartment of hematopoietic progenitor cells as indicated by high expansion of CD34⁺90⁺, a hematopoietic cell subpopulation with long-term SCID-reproliferating activity and high expansion of long-term culture-initiating cells (LTC-IC) was detected after long-term culture for 6 weeks (FIG. 3d). In contrast, the differences in effect, associated with such culture conditions, were not observed when only CD34⁺ cells were cultured without co-culture with MSCs (FIGS. 3b to 3d). In other words, this finding shows that the effects according to different culture conditions are derived from the variations in cultures caused by changes in MSC function, rather than direct effects on HSCs. Such results show that MSCs may exhibit high HSC supporting activity depending on the culture conditions (stimulatory serum or medium), and the high supporting activity with respect to HSC self-renewal is highly associated with the MSC culture conditions exhibiting a high frequency of CFU-Fs (>50) during culture.

### 3-2. Confirmation of Hematopoietic Recovery Effect Using Animal Model

Based on these results, the inventors noted that such a difference in the supporting activity of MSCs could be a factor capable of evaluating variable levels of hematopoietic recovery after co-transplantation of MSCs and HSCs and MSCs were randomly designed to be similar to clinical co-injection (transplantation) of MSCs. A congenic murine repopulation model was used to evaluate the kinetics of engraftment in peripheral blood over 9 to 12 weeks after transplantation. For evaluation of the engraftment of human hematopoietic cells in peripheral blood, mice were prepared, considering the limitations of xenograft animal models due to species specificity of cytokines in murine BM and kinetics of early hematopoietic engraftment of donor-derived cells reaching a plateau.

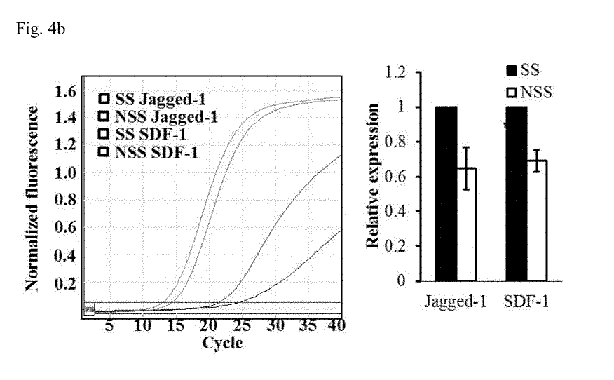

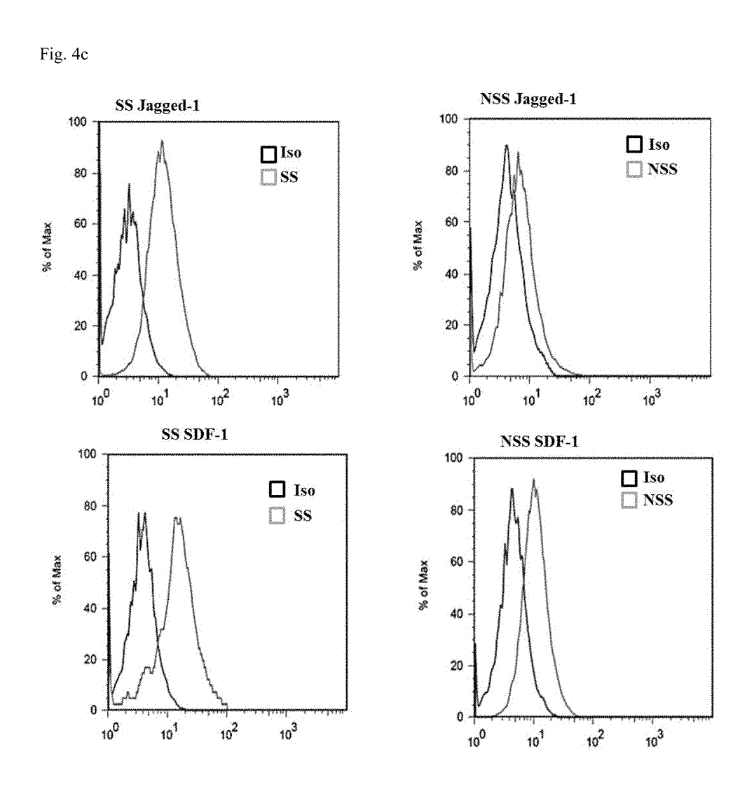

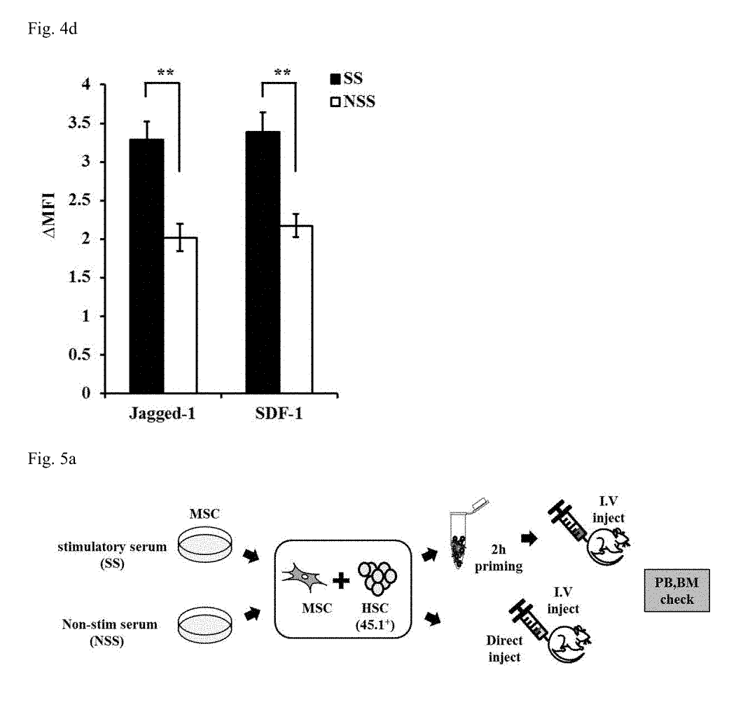

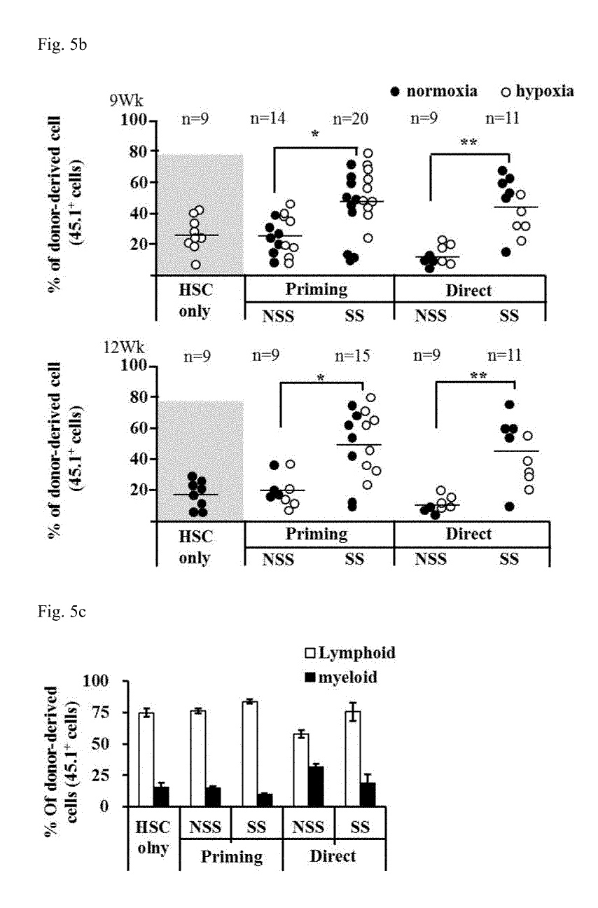

Like human MSCs, in murine BM-MSCs, a significant difference in the CFU-F number under stimulatory or non-stimulatory conditions was observed (FIG. 4a), and in response to a stimulatory or non-stimulatory serum medium, murine BM-MSCs cultured under stimulatory conditions showed higher expression levels of Jagged-1 and SDF-1 than those cultured under non-stimulatory conditions (FIGS. 4b to 4d). Subsequently, MSCs cultured under stimulatory and non-stimulatory culture conditions were co-transplanted with donor HSCs into lethally irradiated recipient mice. Particularly, to exclude the influences of a mixture of HSCs and MSCs due to intercellular contact, the inventors co-transplanted the mixture into recipients by transplantation after 2-hour mixing (priming) or a co-injection of the mixture (direct method) without pre-treatment, and examined the separate effects thereby (FIG. 5a). The co-transplantation of MSCs and HSCs under non-stimulatory conditions did not improve early hematopoietic engraftment in both experimental animals (the priming and direction methods), compared to single transplantation of HSCs, whereas, in the case of co-transplantation of MSCs and HSCs expanded under stimulatory conditions, in the early phase of hematopoietic recovery for 9 to 12 weeks after transplantation, engraftment levels were significantly increased in both experimental animals (FIG. 5b). In addition, co-transplantation of MSCs exposed to hypoxia (1% O₂) for 48 hours before transplantation resulted in a similar difference in engraftment levels between stimulatory and non-stimulatory culture conditions (FIG. 5b). Therefore, it could be seen that the improvement in engraftment is consistently observed both under a normoxic condition or a hypoxic condition.

Noticeably, the enhancement in engraftment was observed without a significant shift in the lympho-myeloid lineage distribution of donor-derived cells, indicating that the increase in repopulation occurred at the level of multi-lineage repopulating cells (FIG. 5c). Supporting these results, in BMs of recipients transplanted with MSCs under stimulatory conditions, a higher number of donor-derived stem cells indicated by a large amount of primitive (Lin⁻Sca-1⁺c-kit⁺) cells was observed in the repopulated BM (FIG. 5d). In other words, these results show that culture conditions inducing the difference in the niche activity of MSCs are highly associated with hematopoietic recovery and heterogeneity in the regeneration of HSCs.

### Example 4. Confirmation of Reversibly Switching Niche Activity of MSCs

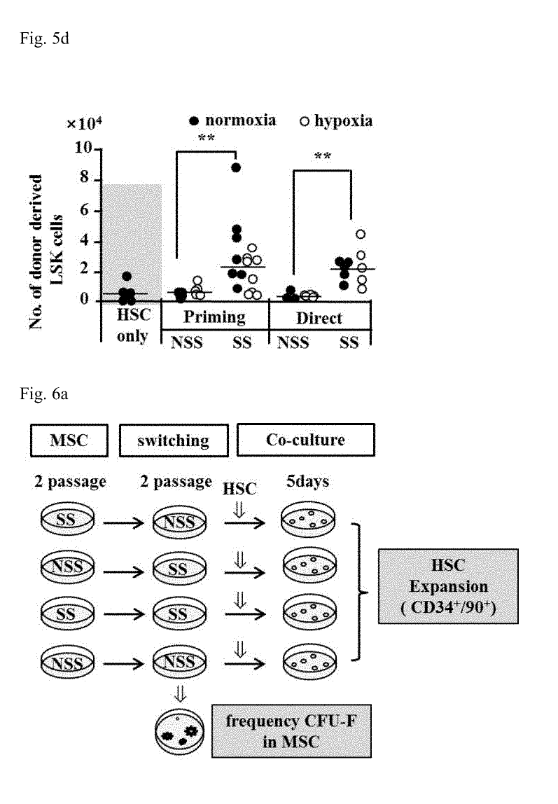

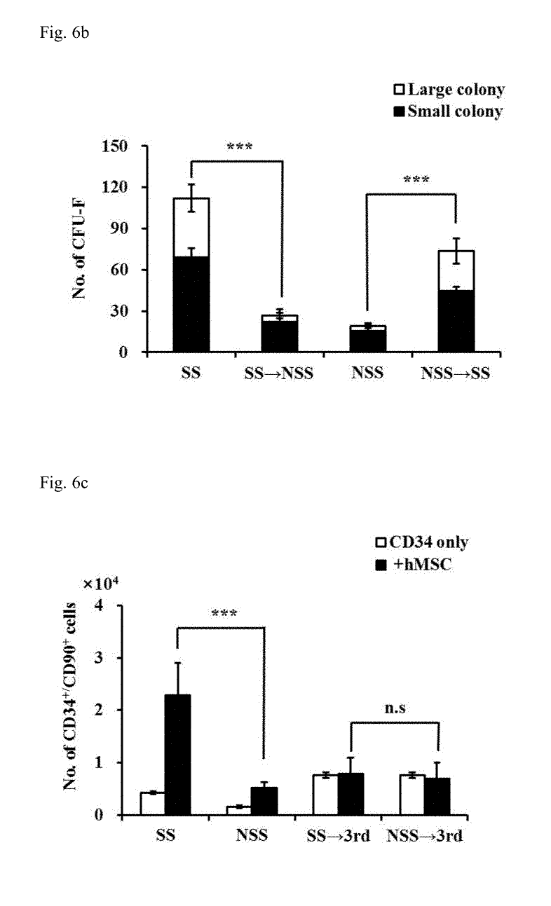

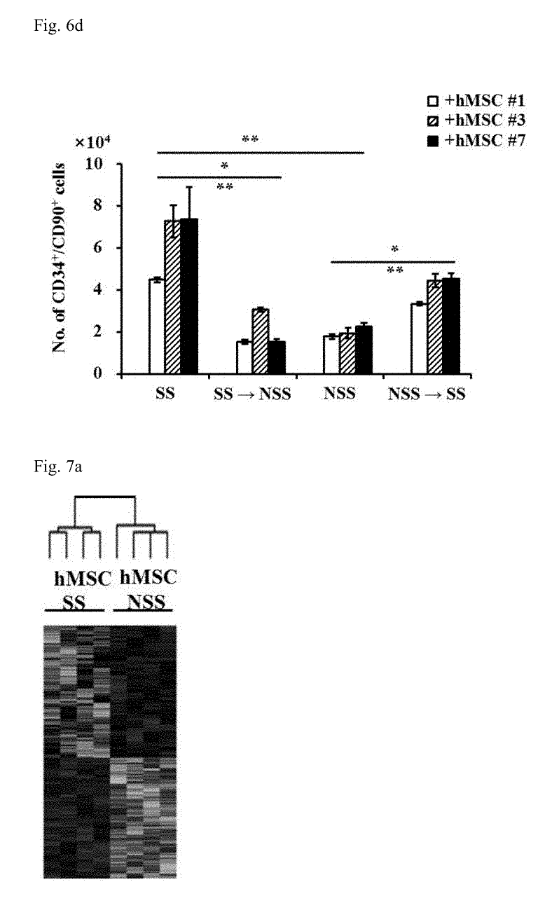

In this example, to express the difference in the HSC supporting activity of MSCs as a function of culture conditions, experimental conditions were set as shown in FIG. 6a. First, the inventors examined whether such differences of MSCs could be attributed to selective expansion of distinct MSC subsets with clonal heterogeneity between the two conditions. To this end, the inventors switched the MSC culture between different culture conditions, and examined their influences on MSC functions.

MSCs grown in an early stage under stimulatory conditions showed a dramatic decrease in CFU-Fs as the conditions were switched to non-stimulatory conditions, whereas MSCs switched from non-stimulatory conditions to stimulatory conditions were again increased in CFU-Fs including highly proliferating (large) colonies (FIG. 6b).

In addition, as a result of confirming the effect of switching culture conditions on the supporting activity of MSCs for HSCs, the difference of stimulatory or non-stimulatory conditions on the expansion of a primitive hematopoietic cell population (CD34⁺90⁺) completely disappeared when the medium was replaced with a third medium (FIG. 6c). Similarly, the effects of stimulatory or non-stimulatory conditions on the CD34⁺90+ expansion were reversibly switched when the culture was under different conditions (FIG. 6d), which showed that the niche activity and hematopoietic recovery effect of MSCs are reversibly switched by the change in culture conditions. In addition, it was seen that the difference in the niche activity of MSCs during the expansion period is caused by the difference in the functional state of MSCs induced by extrinsic factors derived from culture conditions, rather than clonal heterogeneity caused by selective growth of specific subsets.

### Example 5. Confirmation of Signaling Mechanism Regulating MSC Characteristics

In this example, to additionally confirm that the difference in niche activity can be caused by the functional state of MSCs, gene expression profiles were compared by performing microarray for three independent MSCs cultured under stimulatory or non-stimulatory conditions.

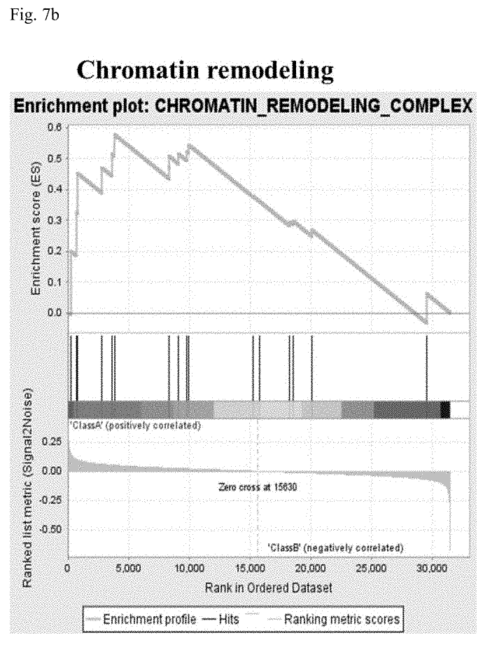

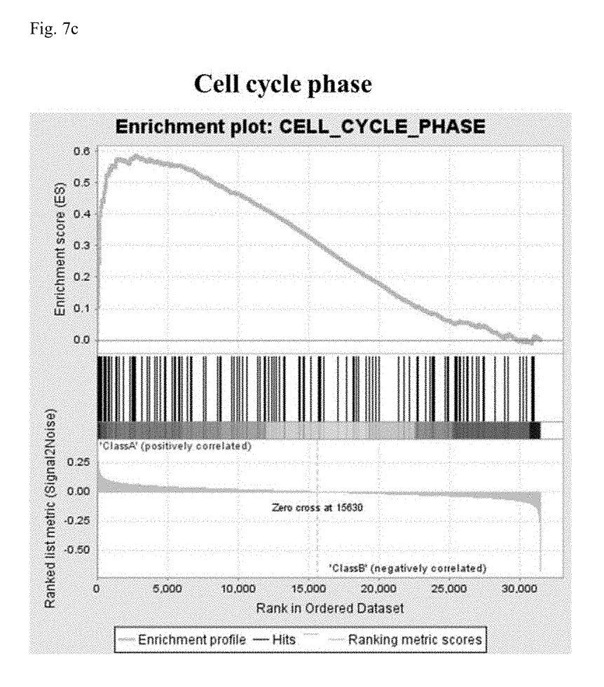

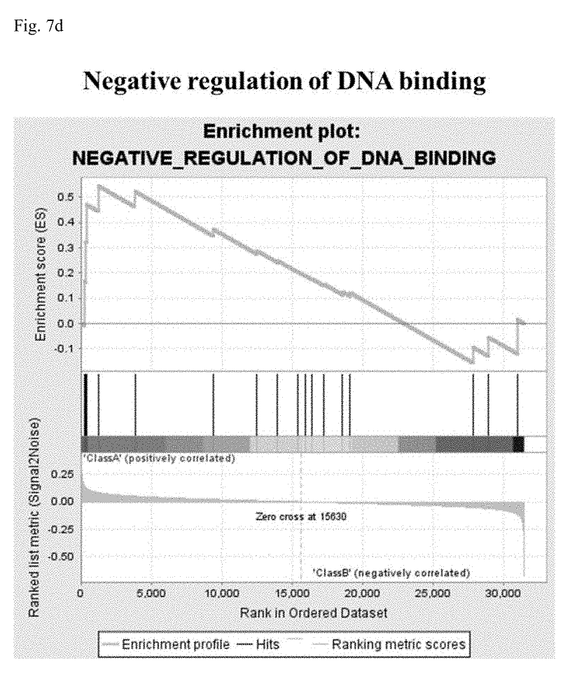

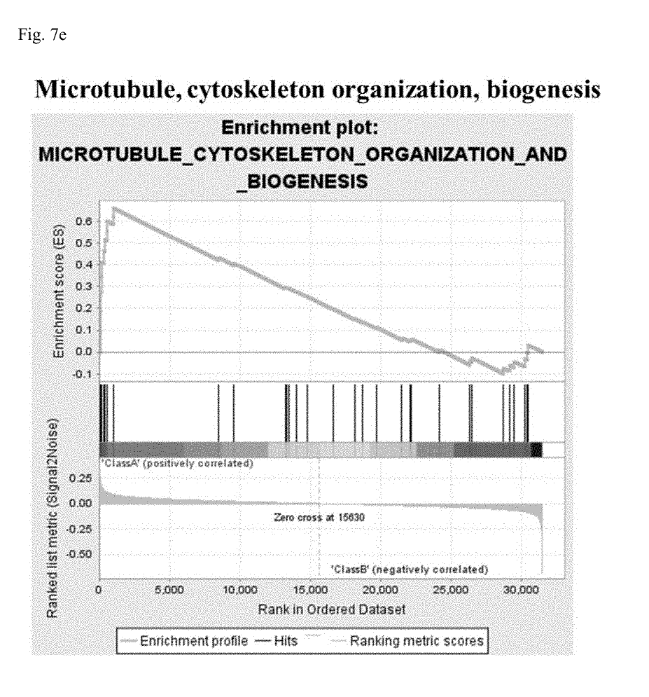

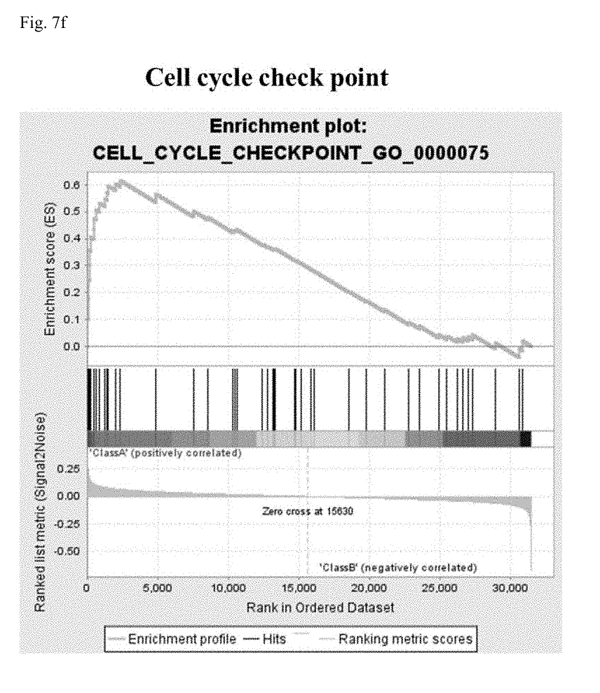

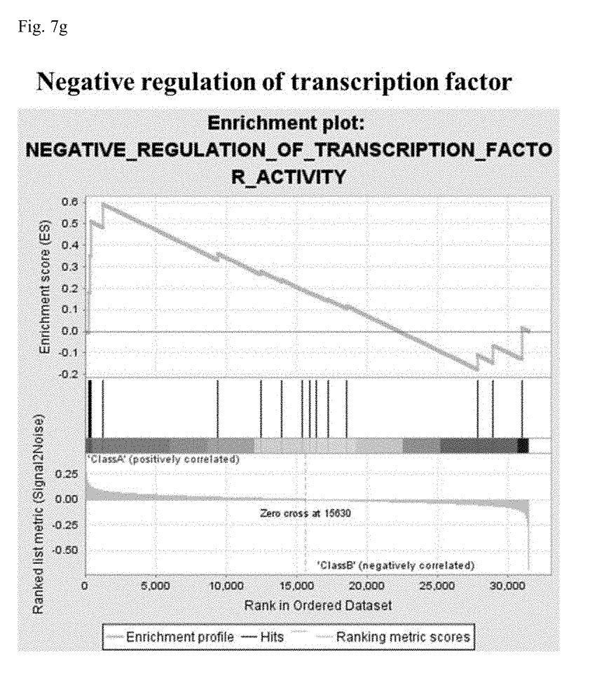

Among a total of 47,323 genes, 785 genes exhibited significant expression differences between the two MSC groups (FIG. 7a). When the difference in gene expression was analyzed as functional annotation by gene set enrichment analysis (GSEA), 15 gene ontology (GO) categories were up-regulated, and 4 GO categories were down-regulated in stimulatory MSCs, compared to non-stimulatory MSCs (FIGS. 7b to 7g, FDR<25%). These results show that the two groups of MSCs are under the unique signaling pathway indeed for a distinctive biological function.

To identify upstream regulators that can cause a transcriptomic difference, the inventors carried out pathway analysis (IPA, Ingenuity Systems, www.ingenuity.com) for differentially expressed gene sets. As a result, five upstream signaling pathways exhibiting most significant changes in downstream targets were identified, and the results are shown in Table 1.

TABLE 1

| Upstream | | Predicted | Activation | p-value of |
|---|---|---|---|---|
| regulato | Molecule type | activation state | z-score | overlap |
| P53 | Transcription | Inhibited | −3.375 | 1.63 × 10⁻¹⁰ |
| | regulator | | | |
| TRIB3 | Kinase | Inhibited | −2.598 | 5.05 × 10⁻⁸ |
| TGFB1 | Growth factor | | −0.43 | 7.5 × 10⁻⁷ |
| RABL6 | Other | Activated | 3.317 | 9.38 × 10⁻⁷ |
| ATF4 | Transcription | Activated | 2.586 | 1.14 × 10⁻⁶ |
| | regulator | | | |

As shown in Table 1, along with multiplicity of tumor-growth factor-signals (TGF-1), the inhibition of p53 and tribbles pseudokinase 3 (TRIB3), and the activation of RAS oncogene family-like 6 (RABL6) and activating transcription factor 4 (ATF4) were confirmed. These results showed that MSCs cultured under stimulatory or non-stimulatory conditions are under unique signaling pathways which may impose different niche activities.

It should be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects, and are not limitative.

What is claimed is:

1. A method of screening mesenchymal stem cells with improved niche activity, comprising:
ex-vivo culturing isolated mesenchymal stem cells; and
selecting mesenchymal stem cells in which 10% or more of a total of the cultured mesenchymal stem cells form colony-forming unit fibroblasts (CFU-Fs).

2. The method according to claim 1, wherein the culturing of mesenchymal stem cells is performed by passaging the mesenchymal stem cells two to five times.

3. The method according to claim 1, wherein the mesenchymal stem cells are derived from human adipose tissue, bone marrow, peripheral blood or umbilical cord blood.

4. The method according to claim 1, wherein the niche activity supports the undifferentiating capacity of hematopoietic stem cells, and stimulates self-renewing capacity.

5. A composition for stimulating self-renewal of hematopoietic stem cells, comprising mesenchymal stem cells selected by the method of claim 1.

6. The composition according to claim 5, wherein the composition is to transplant into a patient with acute leukemia, chronic myelogenous leukemia, myelodysplastic syndrome, lymphoma, multiple myeloma, a germ cell tumor, breast cancer, ovarian cancer, small cell lung cancer, neuroblastoma, aplastic anemia, erythropathy, Gaucher's disease, Hunter syndrome, adenosine deaminase (ADA) deficiency, Wiskott-Aldrich syndrome, rheumatoid arthritis, systemic lupus erythematosus, or multiple sclerosis, or a patient with damaged hematopoietic cells due to chemotherapy or radiation therapy.

7. The composition according to claim 5, wherein the composition is co-transplanted with hematopoietic stem cells.

8. The composition according to claim 7, wherein the hematopoietic stem cells have Lin⁻Sca-1⁺c-kit⁺ (LSK) as a maker for a primitive undifferentiated state.

9. A method of screening a culture condition to improve the niche activity of mesenchymal stem cells, the method comprising:
assessing the colony-forming unit fibroblast (CFU-F) number of ex-vivo cultured mesenchymal stem cells; and
selecting culture conditions under which 10% or more of a total of the ex-vivo cultured mesenchymal stem cells form CFU-Fs.

10. The method according to claim 9, wherein the MSCs are passaged two to five times.

11. The method according to claim 9, wherein the assessing of the CFU-F number is for assessing the CFU-F number 10 to 17 days after cultured stem cells are plated.

12. The method according to claim 9, wherein the mesenchymal stem cells are derived from human adipose tissue, bone marrow, peripheral blood or umbilical cord blood.

13. The method according to claim 9, wherein the niche activity supports the undifferentiating capacity of hematopoietic stem cells, and stimulates self-renewing capacity.
